(19) United States
(12) Patent Application Publication
(10) Pub. No.: US 2011/0251275 A1
(43) Pub. Date: Oct. 13, 2011

(54) OMEGA-3 FATTY ACIDS FOR REDUCTION OF LP-PLA2 LEVELS

(76) Inventors: Roelof M.L. Rongen, Califon, NJ (US); Douglas Kling, Parsippany, NJ (US); Ralph T. Doyle, JR., Milford, NJ (US); Robert A. Shalwitz, Bexley, OH (US)

(21) Appl. No.: 12/987,484

(22) Filed: Jan. 10, 2011

Related U.S. Application Data
(63) Continuation of application No. 12/445,809.
(60) Provisional application No. 60/852,398, filed on Oct. 18, 2006.

(51) Int. Cl. A61K 31/202; A61P 9/00; A61P 9/10; A61K 31/366; A61P 3/06

(52) U.S. Cl. 514/460; 514/560

(57) ABSTRACT

Methods are provided for utilizing omega-3 fatty acids, or a combination of a dyslipidemic agent and omega-3 fatty acids, for the reduction of lipoprotein-associated phospholipase A₂ (Lp-PLA₂) levels. The methods are especially useful in the treatment of patients with primary hypercholesterolemia or hypertriglyceridemia or mixed dyslipidemia, coronary heart disease (CHD), vascular disease, atherosclerotic disease and related conditions, and for the prevention or reduction of major adverse cardiovascular events (MACE), major coronary events (MCE), particularly myocardial infarction (MI), revascularizations and ischemic stroke.

### BACKGROUND OF THE INVENTION

1. Field of the Invention

The present invention relates to a method utilizing administration or a unit dosage of a monotherapy of omega-3 fatty acids or a combination of a dyslipidemic agent and omega-3 fatty acids for the reduction of lipoprotein-associated phospholipase A₂ (Lp-PLA₂) levels. This method is especially useful in the treatment of patients with primary hypertriglyceridemia or hypercholesterolemia or mixed dyslipidemia, coronary heart disease (CHD), vascular disease, atherosclerotic disease and related conditions, and for the prevention or reduction of major adverse cardiovascular events (MACE), major coronary events (MCE), particularly myocardial infarction (MI), revascularizations and ischemic stroke.

2. Description of the Related Art

In humans, cholesterol and triglycerides are part of lipoprotein complexes in the bloodstream, and can be separated via ultracentrifugation into high-density lipoprotein (HDL), intermediate-density lipoprotein (IDL), low-density lipoprotein (LDL) and very-low-density lipoprotein (VLDL) fractions. Cholesterol and triglycerides are synthesized in the liver, incorporated into VLDL, and released into the plasma. High levels of total cholesterol (total-C) and LDL-C promote human atherosclerosis, and decreased levels of HDL-C are associated with the development of atherosclerosis.

Lp-PLA₂ is primarily produced by macrophages, but is also produced by monocytes, T-lymphocytes, and mast cells. See, e.g., Hakkinen, T, et al., Atheroscler Thromb Vasc Biol. 19:2909-2917 (1999). It acts within the walls of blood vessels, and is upregulated in atherosclerosis. Lp-PLA₂ levels do not increase in response to inflammatory cytokines, such as IL-1, IL-6, or TNF-alpha. Lp-PLA₂ travels on LDL and hydrolyzes oxidized LDL particles to generate two highly inflammatory mediators, lysophosphatidylcholine (lyso-PC) and oxidized fatty acid (oxFA). As LDL and HDL decrease, Lp-PLA₂ is reduced. See, e.g., MacPhee, C H, et al., Biochem J. 338:479-487 (1999).

Lipoprotein-associated phospholipase A₂ (Lp-PLA₂) is an inflammatory marker that has been shown to be associated with, and may actively promote inflammation. See, e.g., Hakkinen, T, et al., supra, who demonstrated that Lp-PLA₂ was expressed in macrophages present in atherosclerotic lesions, and exhibited a 6-fold higher activity level in atherosclerotic arteries in a rabbit model. Elevated plasma levels of Lp-PLA₂ have also been correlated with atherosclerosis, coronary heart disease (CHD), and cardiovascular disease (CVD), and have also been associated with an increased risk of stroke.

Most population studies have demonstrated a two-fold risk increase in cardiovascular disease when Lp-PLA₂ is elevated. Packard, C J, et al., N Engl J Med 343:1148-55 (2000) examined blood factors including C-reactive protein, white cells, fibrinogen, and Lp-PLA₂ levels in men with hypercholesterolemia who were enrolled in the West of Scotland Coronary Prevention Study (WOSCOPS), which evaluated the value of pravastatin therapy in the prevention of coronary events. The study found a strong, positive association between Lp-PLA₂ levels and risk of coronary events that was not confounded by other factors. Patients in the highest quintile for Lp-PLA₂ levels had a risk of coronary events that was nearly double that of patients whose Lp-PLA₂ levels were in the lowest quintile. This and other studies demonstrate that although LDL is the lipid value that is still most commonly used to assess the risk of CHD, CVD, and related conditions, Lp-PLA₂ may better reflect risk.

Koenig, W., et al., Circulation 110(14):1903-1908 (2004) noted that although most CHD patients exhibit at least one risk factor, this is not always the case, and sought to investigate the association between C-reactive protein and Lp-PLA₂ in order to more accurately predict the long-term risk of developing CHD, particularly in patients without elevated lipoprotein levels. In a patient population of 45-64 year old men, Lp-PLA₂ levels were found to be positively correlated with age and total cholesterol, but not with smoking, BMI, or systolic blood pressure. Lp-PLA₂ was slightly correlated with HDL-C levels, although it was noted that other studies had found that there was no correlation. Lp-PLA₂ was only marginally correlated with C-reactive protein levels.

However, Blake, G J, et al., J. Am. Coll. Cardiol. 38(5)1302-1306 (2001) found that Lp-PLA₂ was highly correlated with LDL-C, BMI, and HDL-C, and that after adjusting for these risk factors, there was little evidence of of an association with future cardiovascular risk in healthy middle-aged women.

Ballantyne, C M, et al., Circulation 109(7):837-842 (2004) found that Lp-PLA₂ and C-reactive protein may be complementary in identifying individuals, both male and female, who are at high risk for CHD despite low LDL-C levels. No significant associations were observed in individuals with high levels of LDL-C. Ballantyne, et al., Scientific Session of American Heart Association, New Orleans (2004), also evaluated the data from the Atherosclerosis Risk in Communities (ARIC) study and determined that the average baseline level of Lp-PLA₂ in the stroke cohort differed significantly from the non-stroke cohort, despite similar LDL-C levels, and found that the risk of stroke was increased nearly two-fold, even after accounting for other risk factors. In a study published in Arch. Intern. Med. 165:2479-2482 (2005), Ballantyne, at al. also found that found that Lp-PLA₂ and C-reactive protein may also be complementary in identifying individuals at high risk for ischemic stroke, and recommended further study to determine whether selective inhibition of Lp-PLA₂ reduces ischemic stroke, and whether statins and/or fibrates are more effective for stroke prevention in patients with elevated Lp-PLA₂ levels.

Lp-PLA₂ may predict future cardiovascular events in patients already suffering from coronary heart disease. See, e.g., Koenig, W., at al., Arterioscler. Thromb. Vasc. Biol. 26:1586-1593 (2006). However, in patients who had experienced an acute coronary syndrome, such as myocardial infarction or unstable angina, within the previous 30 day period, Lp-PLA₂ levels were not significantly associated with the risk of subsequent cardiovascular events. See, e.g., O'Donoghue, M, et al., Circulation 113(4):1745-1752 (2006). When measured over the course of follow-up treatment, Lp-PLA₂ levels were useful as prognostic factors when considered in addition to the traditional risk factors LDL-C and C-reactive protein.

In pre-clinical animal studies, inhibition of Lp-PLA₂ has been found to attenuate the inflammatory process and slow atherosclerotic progression. See, e.g., Hakkinen, T, et al., supra. In human studies, both statins and fibrates have been shown to lower Lp-PLA₂ levels. Packard, et al., supra, demonstrated a 17% reduction in Lp-PLA₂ levels when patients were treated with pravastatin. Tsimihodimos, et al., J Lipid Res 43: 256-263 (2002) demonstrated a reduction of Lp-PLA₂ by 22-28% in patients treated with fenofibrate, and a reduction of Lp-PLA₂ by 28-42% in patients treated with atorvastatin. Muhleshtein, et al., J Am Coll Cardiol 48:396-401 (2006) demonstrated that simvastatin alone lowered Lp-PLA₂ levels by 25%. O'Donoghue, M, et al., supra, demonstrated that atorvastatin reduced Lp-PLA₂ levels by 20%, whereas Lp-PLA₂ levels rose 3.6% with pravastatin.

Other compounds are also being developed to inhibit PLA₂ activitiy. U.S. Patent Application Publication No. 2006/0014759 embodiments a method of treating or preventing a disease or disorder in a patient, or preventing progression of symptoms of a disease in a patient, by administering a therapeutic substituted indole compounds that inhibit the activity of various phospholipase enzymes, particularly cytosolic phospholipase A₂ enzymes (cPLA₂). The disease or disorder being treated or prevented may include strokes and atherosclerosis.

Although their effects on Lp-PLA₂ levels have not been studied, various agents, such as dyslipidemic agents and omega-3 fatty acids, have been used as monotherapy to treat hyperlipidemia, hypercholesterolemia and hypertriglyceridemia.

Dyslipidemic agents commonly include HMG-CoA reductase inhibitors (statins), cholesterol absorption inhibitors, niacin and derivatives such as nicotinamide, fibrates, bile acid sequestrants, MTP inhibitors, LXR agonists and/or antagonists and PPAR agonists and/or antagonists.

Statins, which are 3-hydroxy-3-methyl glutaryl coenzyme A (HMG-CoA) reductase inhibitors, have been used to treat hyperlipidemia and arthrosclerosis, for example. Typically, statin monotherapy has been used to treat cholesterol levels, particularly when a patient is not at an acceptable LDL-C level. Statins inhibit the enzyme HMG-CoA reductase, which controls the rate of cholesterol production in the body. Statins lower cholesterol by slowing down the production of cholesterol and by increasing the liver's ability to remove the LDL-cholesterol already in the blood. See, e.g., Ballantyne et al., Am. Heart J. 151(5):975.e1-975.e9 (2006).

Cholesterol absorption inhibitors, such as ezetimibe and MD-0727, are a class of lipid-lowering compounds that selectively inhibit the intestinal absorption of cholesterol. Ezetimibe acts on brush border of the small intestine and decreases biliary and dietary cholesterol from the small intestine uptake into the enterocytes. See, e.g., Orse et al., Effects Of Ezetimibe/simvastatin On Lipoprotein Subclasses In Patients With Primary Hypercholesterolemia, 2006 World Cardiology Congress—poster presentation; and Ballantyne et al., Effects of Ezetimibe/Simvastatin Compared to Simvastatin Monotherapy in Reducing C-Reactive Protein and Low Density Lipoprotein-Cholesterol, 2006 World Cardiology Congress—poster presentation.

Cholesteryl ester transfer protein (CETP) inhibitors, such as torcetrapib, inhibit the CETP molecule which, among other things, moves cholesterol from the HDL form to the LDL form. Inhibiting this molecule is, therefore, a promising approach to increasing HDL cholesterol levels. See, e.g., Bhardwaj et al., Indian J. Pharmacol. 37:46 (2005).

Niacin (nicotinic acid or 3-pyridinecarboxylic acid) has previously been used to treat hyperlipidemia and atherosclerosis. Niacin is known to reduce total cholesterol, LDL-C and triglycerides and increase HDL-C. Niacin therapy is also known to decrease serum levels of apo-B. However, the magnitude of the individual lipid and lipoprotein response from niacin therapy may be influenced by the severity and type of underlying lipid abnormality. See, e.g., McKenney et al. Atherosclerosis 7(suppl):174. Abstract Tu-W27:4 (2006).

Fibrates such as fenofibrate, bezafibrate, clofibrate and gemfibrozil, are PPAR-alpha agonists and are used in patients to decrease lipoproteins rich in triglycerides, to increase HDL and to decrease atherogenic-dense LDL. Fibrates are typically orally administered to such patients.

Fenofibrate or 2-[4-(4-chlorobenzoyl)phenoxy]-2-methyl-propanoic acid, 1-methylethyl ester, which belongs to the fibrate family, has been known for many years as a medicinal active principle because of its efficacy in lowering blood triglyceride and cholesterol levels. Fenofibrate is an active principle which is very poorly soluble in water and the absorption of fenofibrate in the digestive tract is limited. A treatment of 40 to 300 mg of fenofibrate per day enables a 20 to 25% reduction of cholesterolemia and a 40 to 50% reduction of triglyceridemia to be obtained.

Bile acid sequestrants, such as cholestyramine, colestipol and colesevelam, are a class of drugs that binds bile acids, prevents their reabsorption from the digestive system, and reduces cholesterol levels. The usual effect of bile acid sequestrants is to lower LDL-cholesterol by about 10 to 20 percent. Small doses of sequestrants can produce useful reductions in LDL-cholesterol. See, e.g., Bard et al., Am. J. Cardiol., 76(2): 65A-70A (2005).

MTP inhibitors, such as implitapide, are known to inhibit the secretion of cholesterol and triglycerides.

Liver X receptors (LXRs) are “cholesterol sensors” that regulate the expression of genes involved in lipid metabolism in response to specific oxysterol ligands (Repa et al., Annu. Rev. Cell Dev. Biol. 16: 459-481(2000)). LXR agonists and antagonists are potential therapeutic agents for dyslipidemia and atherosclerosis.

PPAR-gamma agonists, such as the thiazolidinediones pioglitazone and rosiglitazone, have been shown to improve surrogate markers of cardiovascular risk and atherosclerosis. For example, thiazolidinediones decrease C-reactive protein and carotid intima-media thickness. Non-thiazolidinediones, such as tesaglitazar, naviglitizar and muraglitazar, are dual alpha/gamma PPAR agonists. These compounds are used for lowering glucose, insulin, triglycerides and free fatty acids.

Partial PPAR-gamma agonist/antagonists, such as metaglidasen, are used for the treatment of type II diabetes.

Marine oils, also commonly referred to as fish oils, are a good source of two omega-3 fatty acids, eicosapentaenoic acid (EPA) and docosahexaenoic acid (DHA), which have been found to regulate lipid metabolism. Omega-3 fatty acids have been found to have beneficial effects on the risk factors for cardiovascular diseases, especially mild hypertension, hypertriglyceridemia and on the coagulation factor VII phospholipid complex activity. Omega-3 fatty acids lower serum triglycerides, increase serum HDL-cholesterol, lower systolic and diastolic blood pressure and the pulse rate, and lower the activity of the blood coagulation factor VII-phospholipid complex. Further, omega-3 fatty acids seem to be well tolerated, without giving rise to any severe side effects.

One such form of omega-3 fatty acid is a concentrate of omega-3, long chain, polyunsaturated fatty acids from fish oil containing DHA and EPA and was sold under the trademark OMACOR® and is now known as LOVAZA™. Such a form of omega-3 fatty acid is described, for example, in U.S. Pat. Nos. 5,502,077, 5,656,667 and 5,698,594, each incorporated herein by reference.

Patients with mixed dyslipidemia or hypercholesteremia often present with blood levels of LDL cholesterol greater than 190 mg/dl, triglyceride levels of 200 mg/dl or higher, and Lp-PLA₂ levels of greater than 350 μg/l. The use of diet and single-drug therapy does not always decrease LDL cholesterol, triglycerides and/or Lp-PLA₂ levels adequately enough to reach targeted values in patients with mixed dyslipidemia or hypercholesterolemia. In these patients, therapy using omega-3 fatty acids as monotherapy, or a complementary combination therapy of a dyslipidemic agent and omega-3 fatty acids, may be desirable to reduce Lp-PLA₂ levels.

Studies have examined the combined effects of omega-3 fatty acids (obtained by supplementation or dietary modification) and statin therapy. See, e.g., Hong et al., Chin. Med. Sci. J. 19:145-49 (2004); Contacos et al., Arterioscl. Thromb. 13:1755-62 (1993); Hansen et al., Arterioscl. Thromb. 14(2): 223-229 (February 1994); Nordoy et al., Nutr. Metab. Cardiovasc. Dis. (2001) 11:7-16; Durrington et al., Heart, 85:544-548 (2001). None of these studies report on the effects of treatment with omega-3 fatty acids, or treatment with a combination of a dyslipidemic agent and omega-3 fatty acids, on Lp-PLA₂ levels.

There is clearly a great need in the art for compositions that are useful for reducing Lp-PLA₂ levels, particularly in patients with primary hypertriglyceridemia or hypercholesterolemia or mixed dyslipidemia, coronary heart disease (CHD), vascular disease, atherosclerotic disease and related conditions, and for the prevention or reduction of cardiovascular and vascular events in patients at risk thereof, as well as methods for making such compositions. Methods of reducing Lp-PLA₂ levels and treating dyslipidemia, using the formulations are also needed, particularly in patients with primary hypertriglyceridemia or hypercholesterolemia or mixed dyslipidemia, coronary heart disease (CHD), vascular disease, atherosclerotic disease and related conditions, and in patients at risk of suffering cardiovascular and vascular events, such as strokes.

### SUMMARY OF THE INVENTION

There is an unmet need in the art for methods for the reduction of Lp-PLA₂ levels with omega-3 fatty acids, either as monotherapy or in combination therapy with a dyslipidemic agent where there is significant reduction over monotherapy with the dyslipidemic agent alone. This method is especially useful in the treatment of subjects such as human patients with primary hypercholesterolemia or hypertriglyceridemia or mixed dyslipidemia, coronary heart disease (CHD), vascular disease, atherosclerotic disease and related conditions, and for the prevention or reduction of major adverse cardiovascular events (MACE), major coronary events (MCE), particularly myocardial infarction (MI), revascularizations and ischemic stroke.

Some embodiments of the present invention provide for a method of utilizing omega-3 fatty acids, or a combination of a dyslipidemic agent and omega-3 fatty acids, for the reduction of Lp-PLA₂ levels, which is suitable for the treatment of primary hypercholesterolemia, hypertriglyceridemia, or mixed dyslipidemia, coronary heart disease, vascular disease, atherosclerotic disease and related conditions, and the prevention or reduction of major adverse cardiovascular events (MACE), major coronary events (MCE), particularly myocardial infarction (MI) and ischemic stroke.

Some embodiments according to the present invention include a method of lipid therapy in a subject in need thereof comprising administering to the subject an effective amount of omega-3 fatty acids for reducing an Lp-PLA₂ level, or a combination of an effective amount of a dyslipidemic agent and omega-3 fatty acids wherein an Lp-PLA₂ level in the subject is reduced as compared to treatment with the dyslipidemic agent alone.

In other embodiments, the present invention includes methods of lipid therapy in a subject group in need thereof comprising administering to the subject group an effective amount of omega-3 fatty acids for reducing an Lp-PLA₂ level, or a combination of an effective amount of a dyslipidemic agent and omega-3 fatty acids wherein after administration to the subject group an Lp-PLA₂ level of the subject group is reduced as compared to a control group treated with the dyslipidemic agent alone. In preferred embodiments, the subject group has at least one of the following conditions: primary hypertriglyceridemia or hypercholesterolemia or mixed dyslipidemia. In other preferred embodiments, the method is useful for the prevention or reduction of major adverse cardiovascular events (MACE), major coronary events (MCE), particularly myocardial infarction (MI), revascularizations and ischemic stroke. In yet other embodiments, the subject or subject group has relatively high baseline Lp-PLA₂ as compared to the average healthy population.

One embodiment is directed to a method of lipid therapy, comprising:

determining the baseline Lp-PLA₂ level in a subject or subject group;

administering to the subject or the subject group an effective amount of omega-3 fatty acids, wherein the subject or subject group has an elevated Lp-PLA₂ level; and

reducing the Lp-PLA₂ level as compared to the baseline Lp-PLA₂ level in the subject or subject group.

Another embodiment is directed to a method of lipid therapy, comprising:

determining the baseline Lp-PLA₂ level in a subject or subject group;

administering to the subject or the subject group an effective amount of a combination of omega-3 fatty acids and a dyslipidemic agent, wherein the subject or subject group has an elevated Lp-PLA₂ level; and

reducing the Lp-PLA₂ level as compared to the baseline Lp-PLA₂ level in the subject or subject group or as compared to Lp-PLA₂ level in subjects administered the dyslipidemic agent alone.

In further embodiments, the dyslipidemic agent and the omega-3 fatty acid are administered as a single pharmaceutical composition as a combination product, for example, a unit dosage, comprising the dyslipidemic agent and the omega-3 fatty acids.

In preferred embodiments the pharmaceutical composition(s) comprise LOVAZA™ omega-3 fatty acids, as described in U.S. Pat. Nos. 5,502,077, 5,656,667 and 5,698,594. In other preferred embodiments the pharmaceutical composition(s) comprise omega-3 fatty acids present in a concentration of at least 40% by weight as compared to the total fatty acid content of the composition(s).

In still other preferred embodiments the omega-3 fatty acids comprise at least 50% by weight of EPA and DHA as compared to the total fatty acid content of the composition, and the EPA and DHA are in a weight ratio of EPA:DHA of from 99:1 to 1:99, preferably from 1:2 to 2:1.

In variations of the present invention in which the compositions include a dyslipidemic agent, the dyslipidemic agent is a statin including, but not limited to, pitavastatin, simvastatin, rosuvastatin, pravastatin, atorvastatin, lovastatin and fluvastatin. In preferred embodiments, the statin used in combination with omega-3 fatty acids is simvastatin.

In one aspect of the invention, omega-3 fatty acids, or a combination of a dyslipidemic agent and omega-3 fatty acids, is used in the treatment of subjects with primary hypertriglyceridemia or hypercholesterolemia or mixed dyslipidemia or for the prevention or reduction of major adverse cardiovascular events (MACE), major coronary events (MCE), particularly myocardial infarction (MI), revascularizations and ischemic stroke.

In yet further preferred embodiments of the present invention, the triglyceride levels in the serum of the subject (or the subject group) prior to the first administration of the composition(s) of the present invention (i.e., at “baseline”) is about 200 to about 499 mg/dl.

The invention also includes the use of an effective amount of omega-3 fatty acids, or an effective amount of a combination of a dyslipidemic agent and omega-3 fatty acids, for the manufacture of a medicament useful for any of the treatment methods indicated herein.

Other features and advantages of the present invention will become apparent to those skilled in the art upon examination of the following or upon learning by practice of the invention.

### DETAILED DESCRIPTION OF THE PREFERRED EMBODIMENTS

The present invention is directed to the utilization of omega-3 fatty acids, optionally in combination with a dyslipidemic agent, for reduction of Lp-PLA₂ levels. Preferably, in combination therapy the reduction in Lp-PLA₂ levels is beyond that which is obtained by treatment with a dyslipidemic agent alone. The methods of the present invention are especially useful for the treatment of primary hypertriglyceridemia or hypercholesteremia or mixed dyslipidemia, coronary heart disease, vascular disease, atherosclerotic disease and related conditions. The present invention is also directed to the prevention or reduction of major adverse cardiovascular events (MACE), major coronary events (MCE), particularly myocardial infarction (MI), revascularizations and ischemic stroke, by administering omega-3 fatty acids, optionally in combination with a dyslipidemic agent.

In a typical embodiment, a subject or subject group is tested for baseline Lp-PLA₂ levels, and if the baseline Lp-PLA₂ levels are elevated, the subject or subject group is administered the omega-3 fatty acids, optionally in combination with a dyslipidemic agent, in an amount that reduces the Lp-PLA₂ levels, relative to the baseline level, or the Lp-PLA₂ level achieved when the subject or subject group is administered the dyslipidemic agent alone.

In one embodiment of the present invention, a subject or subject group has a baseline Lp-PLA₂ level above about 300 μg/L, and the use of the invention reduces the Lp-PLA₂ levels to less than about 300 μg/L. According to another embodiment, a subject or subject group has a baseline Lp-PLA₂ level of from about 250-300 μg/L, and the Lp-PLA₂ levels are reduced to less than about 250 μg/L. According to yet another embodiment, a subject or subject group has a baseline Lp-PLA₂ level of from about 200-250 μg/L, and the Lp-PLA₂ levels are reduced to less than about 200 μg/L. Because Lp-PLA₂ is still an emerging marker for cardiovascular disease, levels of Lp-PLA₂ that are considered “elevated” may change with time. The present invention is dependent only on the determination that the levels are “elevated” in accordance with the scientific understanding at the time, and not on the numerical figures.

In some embodiments, the invention provides a novel combination. In a preferred embodiment, the combination comprises omega-3 fatty acids and a dyslipidemic agent, wherein the omega-3 fatty acids are administered simultaneous to administration of the dyslipidemic agent, e.g., as a single fixed dosage pharmaceutical composition or as separate compositions administered at the same time.

In other preferred embodiments, the administration comprises omega-3 fatty acids and a dyslipidemic agent, wherein the omega-3 fatty acids are administered apart from the administration of the dyslipidemic agent, but in a concomitant treatment regime. For example, the dyslipidemic agent may be administered weekly and the omega-3 fatty acids may be administered daily. One skilled in the art with the benefit of the present disclosure will understand that the precise dosage and schedule for the administration of the omega-3 fatty acids and the dyslipidemic agent will vary depending on numerous factors, such as, for example, the route of administration and the seriousness of the condition.

In preferred embodiments, the present invention includes a method of lipid therapy in a subject group comprising administering to the subject group an effective amount of an omega-3 fatty acid for reducing an Lp-PLA₂ level, or a combination of an effective amount of a dyslipidemic agent and an omega-3 fatty acid, wherein the subject group has an elevated baseline triglyceride level above 150 mg/dl, such as 200 to 499 mg/dl, and wherein after administration to the subject group the triglyceride level and an Lp-PLA₂ level of the subject group are reduced as compared to a control group treated with the dyslipidemic agent alone, and an HDL-C level of the subject group is increased as compared to a control group treated with the dyslipidemic agent alone and/or as compared to baseline.

In other preferred embodiments, the present invention includes a method of blood lipid therapy in a subject group comprising administering to the subject group an effective amount of an omega-3 fatty acid for reducing an Lp-PLA₂ level, or a combination of an effective amount of a dyslipidemic agent and an omega-3 fatty acid, wherein the subject group has an elevated baseline triglyceride level above 150 mg/dl, such as 200 to 499 mg/dl and wherein after administration to the subject group the triglyceride level and an Lp-PLA₂ level of the subject group are reduced as compared to a control group treated with the dyslipidemic agent alone, without increasing LDL-C more than 1% as compared to baseline.

In other preferred embodiments, the present invention includes a method of lipid therapy in a subject group, comprising administering to the subject group an effective amount of an omega-3 fatty acid for reducing an Lp-PLA₂ level, or a combination of an effective amount of a dyslipidemic agent and an omega-3 fatty acid, wherein after administration to the subject group a non-HDL-C level, a total cholesterol level, a triglyceride level, and an Lp-PLA₂ level of the subject group is reduced as compared to a control group treated with the dyslipidemic agent alone, and an HDL-C level of the subject group is increased as compared to a control group treated with the dyslipidemic agent alone, without increasing LDL-C more than 1% as compared to baseline.

In other preferred embodiments, the present invention includes a method of lipid therapy in a subject group comprising administering to the subject group an effective amount of an omega-3 fatty acid for reducing an Lp-PLA₂ level, or a combination of a dyslipidemic agent and an omega-3 fatty acid, wherein the subject group has an elevated baseline triglyceride level above 150 mg/dl, such as 200 to 499 mg/dl and wherein after administration to the subject group a non-HDL-C level of the subject group is reduced as compared to a control group treated with the dyslipidemic agent alone.

In still further preferred embodiments, the present invention may be used to treat a subject group for a condition selected from the group consisting of primary hypertriglyceridemia, hypercholesteremia or mixed dyslipidemia, coronary heart disease, vascular disease, and atherosclerotic disease. The methods of the present invention may also be used to prevent or reduce the incidence major adverse cardiovascular events (MACE) or major coronary events (MCE) in a subject group. MACE includes cardiac death, MCE (which includes non-fatal MI or revascularizations (e.g., CABG and angioplasty with or without a stent)), hospitalization for unstable angina, stroke, transient ischemic attack (TIA) and hospitalization for peripheral artery disease (PAD). According to particularly preferred embodiments, the MACE/MCE being prevented or reduced is an MI, revascularizations or an ischemic stroke.

The phrase “compared to treatment with dyslipidemic agent alone” can refer to treatment of the same subject or subject group, or treatment of a comparable subject or subject group (i.e., subject(s) within the same class with respect to a particular blood protein or lipid, such as a cholesterol or triglyceride level) in a different treatment group. The terms “reduce” and “increase” in accordance with the present methods are intended to mean a statistically significant reduction or increase in accordance with its general and customary meaning, i.e., a probability of chance of 5% or less (p=0.05 or less), preferably p=0.025 or less. In some embodiments of the invention, the dyslipidemic agent alone statistically significantly reduces or increases certain levels (such as reducing Lp-PLA₂ levels), and therapy using an omega-3 fatty acid further statistically significantly reduces or increases the levels. The therapies herein may be used by a subject or subject group in need thereof. The term “in need thereof” refers to a subject or subject group which practices the therapy for the stated purpose, such as following the prescribing information for the therapy.

The methods and compositions of the invention may also be used to reduce any of the following lipid levels in a treated subject or subject group, either as monotherapy or in combination as compared to treatment with the dyslipidemic agent alone: non-HDL-C levels, triglyceride levels, VLDL-C levels, total C levels, remnant-like particle cholesterol (RLP-C) levels, apolipoprotein-B (Apo-B) levels and/or apolipoprotein-C3 (Apo-C3) levels. The methods and compositions of the invention, either as monotherapy or in combination as compared to treatment with the dyslipidemic agent alone, may also be used to increase HDL-C levels.

Preferably, non-HDL-C levels may be reduced at least about 5%, preferably at least about 7%, from baseline and/or at least about 5%, preferably at least about 7%, further than treatment with the dyslipidemic agent alone.

Preferably, the triglyceride levels may be reduced by at least about 20%, preferably at least about 25%, as compared to baseline and/or at least about 15%, preferably at least about 20%, further than treatment with the dyslipidemic agent alone.

Preferably, the VLDL-C levels may be reduced by at least about 20%, preferably at least about 25%, as compared to baseline and/or at least about 15%, preferably at least about 20%, further than treatment with the dyslipidemic agent alone.

Preferably, the total C levels may be reduced by at least about 3%, preferably at least about 5%, as compared to baseline and/or at least about 2%, preferably at least about 3%, further than treatment with the dyslipidemic agent alone.

Preferably, the RLP-C levels may be reduced by at least about 20%, preferably at least about 25%, as compared to baseline and/or at least about 15%, preferably at least about 20%, further than treatment with the dyslipidemic agent alone.

Preferably, the Lp-PLA₂ levels may be reduced by at least about 7%, preferably at least about 10%, as compared to baseline and/or at least about 5%, preferably at least about 7%, further than treatment with the dyslipidemic agent alone.

Preferably, the Apo-B levels may be reduced by at least about 3%, preferably at least about 4%, as compared to baseline and/or at least about 1%, preferably at least about 2%, further than treatment with the dyslipidemic agent alone.

Preferably, the Apo-C3 levels may be reduced by at least about 5%, preferably at least about 7%, as compared to baseline and/or at least about 8%, preferably at least about 10%, further than treatment with the dyslipidemic agent alone.

Preferably, the HDL-C levels may be increased by at least about 2%, preferably at least about 3%, as compared to baseline and/or at least about 3%, preferably at least about 5%, further than treatment with the dyslipidemic agent alone.

Preferably, the present invention also decreases the ratio of total cholesterol to HDL-C, preferably by at least about 5%, more preferably at least about 10%, as compared to baseline and/or at least about 5%, preferably at least about 10%, further than treatment with the dyslipidemic agent alone.

According to certain embodiments in which a combination of a dyslipidemic agent and an omega-3 fatty acid are administered, the present invention may incorporate now known or future known dyslipidemic agents in an amount generally recognized as safe.

Preferred dyslipidemic agents include HMG-CoA reductase inhibitors including statins, cholesterol absorption inhibitors such as but not limited to ezetimibe, niacin and derivatives such as nicotinamide, CETP inhibitors such as but not limited to torcetrapib, fibrates such as but not limited to fenofibrate, bezafibrate, clofibrate and gemfibrozil, bile acid sequestrants such as but not limited to cholestyramine, colestipol and colesevelam, MTP inhibitors such as but not limited to those disclosed in WO 00/38725 and Science, 282, 23 October 1998, pp. 751-754, herein incorporated by reference, LXR agonists and/or antagonists, and PPAR agonists and antagonists (such as but not limited to PPAR-alpha, PPAR-gamma, PPAR-delta, PPAR-alpha/gamma, PPAR-gamma/delta, PPAR-alpha/delta, and PPAR-alpha/gamma/delta agonists, antagonists and partial agonists and/or antagonists) such as but not limited to the thiazolidinediones, the non-thiazolidinediones and metaglidasen. There are currently six statins that are widely available: atorvastatin, rosuvastatin, fluvastatin, lovastatin, pravastatin, and simvastatin. A seventh statin, pitavastatin, is in clinical trials. An eighth statin, cerivastatin, has been removed from the U.S. market at the time of this writing. However, it is conceivable to one skilled in the art that cerivastatin may be used in conjunction with some embodiments of the present invention if cerivastatin is ultimately determined to be safe and effective.

Generally, when provided, the effect of the dyslipidemic agent is dose dependent, i.e., the higher the dose, the greater the therapeutic effect. However, the effect of each dyslipidemic agent is different, and therefore the level of therapeutic effect of one dyslipidemic agent cannot be necessarily be directly correlated to the level of therapeutic effects of other dyslipidemic agents. However, those of ordinary skill in the art would understand the correct dosage to be given to a particular subject, based on experience and the seriousness of the condition.

As used herein, the term “omega-3 fatty acids” includes natural or synthetic omega-3 fatty acids, or pharmaceutically acceptable esters, derivatives, conjugates (see, e.g., Zaloga et al., U.S. Patent Application Publication No. 2004/0254357, and Horrobin et al., U.S. Pat. No. 6,245,811, each hereby incorporated by reference), precursors or salts thereof and mixtures thereof. Examples of omega-3 fatty acid oils include but are not limited to omega-3 polyunsaturated, long-chain fatty acids such as a eicosapentaenoic acid (EPA), docosahexaenoic acid (DHA), and α-linolenic acid; esters of omega-3 fatty acids with glycerol such as mono-, di- and triglycerides; and esters of the omega-3 fatty acids and a primary, secondary or tertiary alcohol such as fatty acid methyl esters and fatty acid ethyl esters. Preferred omega-3 fatty acid oils are long-chain fatty acids such as EPA or DHA, triglycerides thereof, ethyl esters thereof and mixtures thereof. The omega-3 fatty acids or their esters, derivatives, conjugates, precursors, salts and mixtures thereof can be used either in their pure form or as a component of an oil such as fish oil, preferably purified fish oil concentrates. Commercial examples of omega-3 fatty acids suitable for use in the invention include Incromega F2250, F2628, E2251, F2573, TG2162, TG2779, TG2928, TG3525 and E5015 (Croda International PLC, Yorkshire, England), and EPAX6000FA, EPAX5000TG, EPAX4510TG, EPAX2050TG, K85TG, K85EE, K80EE and EPAX7010EE (EPAX a.s., Lysaker, Norway).

Preferred compositions include omega-3 fatty acids as recited in U.S. Pat. Nos. 5,502,077, 5,656,667 and 5,698,694, which are hereby incorporated herein by reference in their entireties.

Another preferred composition includes omega-3 fatty acids present in a concentration of at least 40% by weight, preferably at least 50% by weight, more preferably at least 60% by weight, still more preferably at least 70% by weight, most preferably at least 80% by weight, or even at least 90% by weight. Preferably, the omega-3 fatty acids comprise at least 50% by weight of EPA and DHA, more preferably at least 60% by weight, still more preferably at least 70% by weight, most preferably at least 80%, such as about 84% by weight. Preferably the omega-3 fatty acids comprise about 5 to about 100% by weight, more preferably about 25 to about 75% by weight, still more preferably about 40 to about 55% by weight, and most preferably about 46% by weight of EPA. Preferably the omega-3 fatty acids comprise about 5 to about 100% by weight, more preferably about 25 to about 75% by weight, still more preferably about 30 to about 60% by weight, and most preferably about 38% by weight of DHA. All percentages above are by weight as compared to the total fatty acid content in the composition, unless otherwise indicated. The percentage by weight may be based on the free acid or ester forms, although it is preferably based on the ethyl ester form of the omega-3 fatty acids even if other forms are utilized in accordance with the present invention.

The EPA:DHA ratio may be from 99:1 to 1:99, preferably 4:1 to 1:4, more preferably 3:1 to 1:3, most preferably 2:1 to 1:2. The omega-3 fatty acids may comprise pure EPA or pure DHA.

The omega-3 fatty acid composition optionally includes chemical antioxidants, such as alpha tocopherol, oils, such as soybean oil and partially hydrogenated vegetable oil, and lubricants such as fractionated coconut oil, lecithin and a mixture of the same.

The most preferred form of omega-3 fatty acids is the LOVAZA™ omega-3 acid (K85EE, Pronova Biocare A.S., Lysaker, Norway) and preferably comprises the following characteristics (per dosage form):

| | Minimum | Maximum |
|---|---|---|
| Test | Value | Value |
| Eicosapentaenoic acid C20:5 | 430 mg/g | 495 mg/g |
| Docosahexaenoic acid C22:6 | 347 mg/g | 403 mg/g |
| EPA and DHA | 800 mg/g | 880 mg/g |
| Total n-3 fatty acids | 90% (w/w) | |

Both the concentrated omega-3 fatty acid product and the combination product of a dyslipidemic agent and concentrated omega-3 fatty acids may be administered in a capsule, a tablet, a powder that can be dispersed in a beverage, or another solid oral dosage form, a liquid, a soft gel capsule, a coated soft gel capsule (see U.S. patent application Ser. No. 11/716,020, hereby incorporated by reference) or other convenient dosage form such as oral liquid in a capsule, as known in the art. In some embodiments, the capsule comprises a hard gelatin. The products may also be contained in a liquid suitable for injection or infusion. The active ingredients of the present invention may also be administered with a combination of one or more non-active pharmaceutical ingredients (also known generally herein as “excipients”). Non-active ingredients, for example, serve to solubilize, suspend, thicken, dilute, emulsify, stabilize, preserve, protect, color, flavor, and fashion the active ingredients into an applicable and efficacious preparation that is safe, convenient, and otherwise acceptable for use.

The omega-3 fatty acids can be administered in a daily amount of from about 0.1 g to about 10 g, more preferably about 1 g to about 8 g, and most preferably from about 2 g to about 6 g.

When provided, the dyslipidemic agent may be administered in an amount more than, equal to or less than the conventional full-strength dose as a single-administered product. For example, the dyslipidemic agent may be administered in an amount of from 10-100%, preferably about 25-100%, most preferably about 50-80%, of the conventional full-strength dose as a single-administered product. In one embodiment of the present invention, the statin can generally be present in an amount from about 0.5 mg to 320 mg, more preferably from about 1 mg to about 160 mg, and most preferably from about 5 mg to about 80 mg, per gram of omega-3 fatty acid. The daily dose may range from about 2 mg to about 640 mg, preferably about 4 mg to about 160 mg.

In some variations of the present invention, the combination of dyslipidernic agent and the omega-3 fatty acids is formulated into a single administration or unit dosage. In preferred embodiments, a statin is utilized selected from the following group: pitavastatin, atorvastatin, rosuvastatin, fluvastatin, lovastatin, pravastatin, and simvastatin.

Pravastatin, which is known in the market as PRAVACHOL® manufactured by Bristol-Myers Squibb, Princeton, N.J., is hydrophilic. Pravastatin is best absorbed without food, i.e., an empty stomach. The dosage of pravastatin, in the combined administration of concentrated omega-3 fatty acids is preferably from 2.5 to 160 mg, preferably 5 to 120, and more preferably from 10 to 80 mg per dosage of concentrated omega-3 fatty acids. In one variation, the combination product using pravastatin is taken at or around bedtime, e.g., 10 pm.

Lovastatin, which is marketed under the name MEVACOR® by Merck, Whitehouse Station, N.J., is hydrophobic. Unlike pravastatin, lovastatin should be taken with meals and accordingly, in some embodiments, the combination product of concentrated omega-3 fatty acids and lovastatin should be taken with food. The dosage of lovastatin, in the combined administration of concentrated omega-3 fatty acids is preferably from 2.5 to 160 mg, preferably 5 to 120 mg, and more preferably from 10 to 60 mg per dosage of concentrated omega-3 fatty acids.

Simvastatin, which is marketed under the name ZOCOR® by Merck, Whitehouse Station, N.J., is hydrophobic. The dosage of simvastatin, in the combined administration of concentrated omega-3 fatty acids is preferably from 1 to 320 mg per day, preferably 2 to 160 mg, and more preferably from 5 to 80 mg per dosage of concentrated omega-3 fatty acids.

Atorvastatin, which is marketed under the name LIPITOR® by Pfizer, New York, N.Y., is hydrophobic and is known as a synthetic statin. The dosage of atorvastatin, in the combined administration of concentrated omega-3 fatty acids is preferably from 2.5 to 320 mg, preferably 5 to 160 mg, and more preferably from 10 to 80 mg per dosage of concentrated omega-3 fatty acids.

Fluvastatin, which is marketed under the name LESCOL® by Novartis, New York, N.Y., is hydrophilic and is known as a synthetic statin. The dosage of fluvastatin, in the combined administration of concentrated omega-3 fatty acids is from 5 to 320 mg, preferably 10 to 160 mg, and more preferably from 20 to 80 mg per dosage of concentrated omega-3 fatty acids.

Rosuvastatin is marketed under the name CRESTOR® by Astra Zeneca, Wilmington, Del. The dosage of rosuvastatin, in the combined administration of concentrated omega-3 fatty acids is from 1 to 160 mg, preferably 2 to 80 mg, and more preferably from 5 to 40 mg per dosage of concentrated omega-3 fatty acids.

The daily dosages of concentrated omega-3 fatty acids, or a dyslipidemic agent and concentrated omega-3 fatty acids, can be administered together in from 1 to 10 dosages, with the preferred number of dosages from 1 to 4 times a day, most preferred 1 to 2 times a day. The administration is preferably oral administration. Other forms of administration may also be used.

In some embodiments, the formulations of the present invention allow for improved effectiveness of the active ingredient(s) as compared to the formulations in the prior art, In embodiments in which both omega-3 fatty acids and a dyslipidemic agent are provided, one or both are administered as a conventional full-strength dose. In other embodiments, the formulations of the present invention may allow for reduced dosages of the active ingredient(s), as compared to the formulations in the prior art, while still maintaining or even improving upon the effectiveness of the active ingredient(s). In embodiments in which both omega-3 fatty acids and a dyslipidemic agent are provided, one or both may be administered as a reduced dose.

The present combination of a dyslipidemic agent and concentrated omega-3 fatty acids may allow for a greater effect than any expected combined or additive effect of the two drugs alone. Moreover, the combined or additive effect of the two drugs may depend on the initial level of triglycerides in the blood of a subject. For example, the triglyceride level of a subject is generally as normal if less than 150 mg/dL, borderline to high if within about 150-199 mg/dL, high if within about 200-499 mg/dL and very high if 500 mg/dL or higher. The present invention may be used to reduce the triglyceride level of a “very high” down to a “high” or “borderline to high” in less than 48 weeks, preferably within 24 weeks, more preferably within 12 weeks, and most preferably within 8 weeks. The present invention may also be used to reduce the triglyceride level of a “high” down to a “borderline to high” or “normal” in less than 48 weeks, preferably within 24 weeks, more preferably within 12 weeks, and most preferably within 8 weeks.

### EXAMPLE

Clinical study: A Randomized, Double-Blind, Placebo-Controlled Study to Assess the Efficacy and Safety of Combined LOVAZA™ and Simvastatin Therapy in Hypertriglyceridemic Subjects

Patients were initially treated with 40 mg/day simvastatin for 8 weeks, whereupon baseline measurements were taken. Initial treatment was thereafter followed by an additional 8 week treatment with either 4 g/day LOVAZA™ omega-3 fatty acids or placebo, while continuing statin therapy, in a double-blind fashion. 243 patients with median baseline triglyceride levels between 200 and 499 mg/dl and LDL-C≦10% above the NCEP ATP-III goal completed the study. The following values were obtained for various lipid parameters and markers.

| | LOVAZA ™ | LOVAZA ™ | Placebo | Placebo | | |
|---|---|---|---|---|---|---|
| | treatment: | treatment: | treatment: | treatment: | | |
| | median % | mean % | median % | mean % | | |
| | change | change | change | change | | |
| | from | from | from | from | Difference | |
| | baseline | baseline | baseline | baseline | (% median) | p-value |
| non-HDL-C | −9.0% | −7.9% | −2.2% | −1.5% | −6.8% | <0.0001 |
| LDL-C | +0.7% | +3.1% | −2.8% | −1.9% | +3.5% | 0.0522 |
| apo-B | −4.2% | −3.8% | −1.9% | −1.2% | −2.3% | 0.0232 |
| TG | −29.5% | −28.2% | −6.3% | −3.5% | −23.2% | <0.0001 |
| VLDL-C | −27.5% | −23.8% | −7.2% | −4.8% | −20.3% | <0.0001 |
| total C | −4.8% | −4.7% | −1.7% | −1.5% | −3.1% | 0.0013 |
| HDL-C | +3.4% | +4.1% | −1.2% | −1.1% | +4.6% | <0.0001 |
| TC/HDL | −9.6% | −8.0% | −0.7% | +0.1% | −8.9% | <0.0001 |
| RLP-C | −36.0% | −29.0% | −10.6% | +0.1% | −25.4% | <0.0001 |
| Lp-PLA₂ | −12.8% | −10.7% | −4.7% | −1.4% | −8.1% | 0.0019 |
| apo-C3 | −7.8% | −7.1% | +3.9% | +3.8% | −11.7% | 0.0002 |

Additional uses for the compositions and methods of the present invention, beyond treatment of primary hypercholesterolemia or hypertriglyceridemia or mixed dyslipidemia, coronary heart disease, vascular disease, atherosclerotic disease and related conditions, and the prevention or reduction of the incidence of MACE and/or MCE, such as MIs and ischemic stroke, are also envisioned. The compositions may also be beneficially incorporated into preparations for use in the treatment of these and other conditions.

It will, of course, be appreciated that the above description has been given by way of example only and that modifications in detail may be made within the scope of the present invention.

Throughout this application, various patents and publications have been cited. The disclosures of these patents and publications in their entireties are hereby incorporated by reference into this application, in order to more fully describe the state of the art to which this invention pertains.

The invention is capable of considerable modification, alteration, and equivalents in form and function, as will occur to those ordinarily skilled in the pertinent arts having the benefit of this disclosure.

While the present invention has been described for what are presently considered the preferred embodiments, the invention is not so limited. To the contrary, the invention is intended to cover various modifications and equivalent arrangements included within the spirit and scope of the detailed description provided above.

1. A method of lipid therapy, comprising:
determining the baseline Lp-PLA₂ level in a subject or subject group;
administering to the subject or the subject group an effective amount of omega-3 fatty acids, wherein the subject or subject group has an elevated Lp-PLA₂ level; and
reducing the Lp-PLA₂ level as compared to the baseline Lp-PLA₂ level in the subject or subject group.

2. The method of claim 1, wherein the method comprises:
determining the baseline Lp-PLA₂ level in a subject or subject group;
administering to the subject or the subject group an effective amount of a combination of omega-3 fatty acids and a dyslipidemic agent, wherein the subject or subject group has an elevated Lp-PLA₂ level; and
reducing the Lp-PLA₂ level as compared to the baseline Lp-PLA₂ level in the subject or subject group or as compared to Lp-PLA₂ level in subjects administered the dyslipidemic agent alone.

3. The method of claim 1, wherein the subjects have at least one of the following conditions or diseases: hypertriglyceridemia, hypercholesterolemia, mixed dyslipidemia, vascular disease, coronary heart disease, and atherosclerotic disease.

4. The method of claim 1, wherein the omega-3 fatty acids are present in a concentration of at least 40% by weight as compared to the total fatty acid content of the composition.

5. The method of claim 1, wherein the omega-3 fatty acids are present in a concentration of at least 80% by weight as compared to the total fatty acid content of the composition.

6. The method of claim 1, wherein the omega-3 fatty acids comprise about 40% to about 55% by weight of EPA as compared to the total fatty acid content of the composition.

7. The method of claim 1, wherein the omega-3 fatty acids comprise about 30% to about 60% by weight of DHA as compared to the total fatty acid content of the composition.

8. The method of claim 1, wherein omega-3 fatty acids comprise omega-3 polyunsaturated, long-chain fatty acids, esters of omega-3 fatty acids with glycerol, esters of omega-3 fatty acids and a primary, secondary or tertiary alcohol, or mixtures thereof.

9. The method of claim 1, wherein the omega-3 fatty acids comprise EPA and DHA in a ratio of EPA:DHA from 2:1 to 1:2.

10. The method of claim 2, further comprising reducing at least one additional level of the subject or subject group, independently selected from the group consisting of: total cholesterol (TC) level, the triglyceride level, very-low density lipoprotein cholesterol (VLDL-C) level, remnant-like particle cholesterol (RLP-C) level, apolipoprotein-B (Apo-B) level, and apolipoprotein-C3 (Apo-C3) level, as compared to treatment with a dyslipidemic agent alone.

11. The method of claim 2, wherein the omega-3 fatty acids and the dyslipidemic agent are administered together in a unit dose form.

12. A method of lipid therapy, comprising reducing an Lp-PLA₂ level of a subject or subject group in need thereof by administering to the subject or subject group a pharmaceutical composition containing omega-3 fatty acids in an amount sufficient to reduce the Lp-PLA₂ level of the subject or subject group.

13. The method of claim 12, wherein the method comprises administering to a subject or subject group in need thereof a combination of an effective amount of a dyslipidemic agent and omega-3 fatty acids wherein after administration to the subject or subject group an Lp-PLA₂ level of the subject or subject group is reduced as compared to a control subject or group treated with the dyslipidemic agent alone.

14. The method of claim 12, wherein the subjects have at least one of the following conditions or diseases: hypertriglyceridemia, hypercholesterolemia, mixed dyslipidemia, vascular disease, coronary heart disease, and atherosclerotic disease.

15. The method of claim 12, wherein the omega-3 fatty acids are present in a concentration of at least 40% by weight as compared to the total fatty acid content of the composition.

16. The method of claim 12, wherein the omega-3 fatty acids are present in a concentration of at least 80% by weight as compared to the total fatty acid content of the composition.

17. The method of claim 12, wherein the omega-3 fatty acids comprise about 40% to about 55% by weight of EPA as compared to the total fatty acid content of the composition.

18. The method of claim 12, wherein the omega-3 fatty acids comprise about 30% to about 60% by weight of DHA as compared to the total fatty acid content of the composition.

19. The method of claim 12, wherein omega-3 fatty acids comprise omega-3 polyunsaturated, long-chain fatty acids, esters of omega-3 fatty acids with glycerol, esters of omega-3 fatty acids and a primary, secondary or tertiary alcohol, or mixtures thereof.

20. The method of claim 12, wherein the omega-3 fatty acids comprise EPA and DHA in a ratio of EPA:DHA from 2:1 to 1:2.

21. The method of claim 13, further comprising reducing at least one additional level of the subject or subject group, independently selected from the group consisting of: total cholesterol (TC) level, the triglyceride level, very-low density lipoprotein cholesterol (VLDL-C) level, remnant-like particle cholesterol (RLP-C) level, apolipoprotein-B (Apo-B) level, and apolipoprotein-C3 (Apo-C3) level, as compared to treatment with a dyslipidemic agent alone.

22. The method of claim 13, wherein the omega-3 fatty acids and the dyslipidemic agent are administered together in a unit dose form.
